(19) United States
(12) Patent Application Publication
(10) Pub. No.: US 2011/0195478 A1
(43) Pub. Date: Aug. 11, 2011

(54) BLADDER CANCER BIOMARKER AND TEST METHOD USING THE SAME

(76) Inventors: Yi-Ting Chen, Kwei-Shan (TW); Jau-Song Yu, Kwei-Shan (TW); Chien-Lun Chen, Kwei-Shan (TW); Yu-Sun Chang, Linkou Shiang (TW)

(21) Appl. No.: 12/849,988

(22) Filed: Aug. 4, 2010

(30) Foreign Application Priority Data
Feb. 11, 2010 (TW) 099104400

(51) Int. Cl. C12N 9/96; C12N 9/04; C12N 9/08; C12N 9/10; C12N 9/12; C12N 9/18; C12N 9/88; C12N 9/90; C07K 14/47; C07K 14/775; C07K 14/805; C07K 16/00; C07K 14/745; G01N 33/561

(52) U.S. Cl. 435/188; 435/190; 435/192; 435/193; 435/194; 435/197; 435/232; 435/233; 530/350; 530/359; 530/385; 530/387.1; 530/393; 436/516

(57) ABSTRACT

The present invention discloses a bladder cancer biomarker and a test method using the same. The biomarker contains at least one of the mentioned 69 compounds, such as apolipoprotein A1 (APOA1), apolipoprotein A2 (APOA2), peroxiredoxin 2 (PRDX2), heparin cofactor 2 precursor (HCII), and serum amyloid A-4 protein (SAA4), which exist in the urine specimen of a testee. The expression intensity of the biomarker can facilitate diagnosis of bladder cancer and evaluation of aggressiveness and malignancy of bladder cancer. Thereby, the physician can arrange an optimized treatment to achieve the best therapeutic effect.

### BACKGROUND OF THE INVENTION

1. Field of the Invention

The present invention relates to a biomarker and a test method using the same, particularly to a bladder cancer biomarker and a test method using the same.

2. Description of the Related Art

The bladder collects urine generated by kidneys. Bladder cancer is the most common urinary cancer. Therefore, the biomarkers for bladder cancer are eagerly expected in the related field. The American Cancer Society (ACS) estimated that there were 70980 new patients of bladder cancer in 2009 and that 14330 persons die of bladder cancer each year. ACS also estimated that the bladder cancer morbidity is 1/27 for males and 1/85 for females and that 90% of bladder cancer patients are over 55 years old. Earlier diagnosis gets better prognosis for cancer. TNM is the most frequently-used bladder cancer staging system. The TNM staging system published by AJCC/UICC in 1997 is based on three variables: primary tumor (T), regional nodes (N), and metastasis (M). The histological classification of cancer includes low grade (G1), medium grade (G2) and high grade (G3). However, the histological classification of cancer is now revised to only G1 and G2 according to the behaviors of cancer cells. Noninvasive tumors present diversity in the histological classification. Most of invasive tumors belong to the high grade in the histological classification.

About 70% of bladder tumors present superficial lesions. Superficial bladder tumors are mainly treated with TUR (transurethral resection). The immunological therapy (such as BCG vaccine) or chemical therapy (doxorubicin) via intrabladder instillation is used as the auxiliary or preventive therapy to eliminate the residual cancer tissue. The recurrence rate of superficial lesions is 50-80%. About 15% low-grade tumors will evolve into high-grade tumors and present muscular invasion. Eradicating the bladder is one option for treating the muscle-invasive bladder cancer. However, such a treatment causes functional damage. In such a case, a urethral reconstruction is required.

The histological examination is decisive and less sensitive to various factors, particularly for low-grade tumors. However, specific and non-invasive biomarkers can reduce the number per year of the expensive and invasive cystoscopic examinations. The high-specificity and high-sensitivity biomarkers can greatly improve the diagnosis and treatment of bladder cancer. Several candidates of bladder cancer biomarkers, which may be used in initial diagnosis, recurrence detection and therapy evaluation, have been found in urine or bladder cancer cells. However, some of these candidate biomarkers do not present superiority in both sensitivity and specificity, and some of them have not been clinically used in large scale. Therefore, more effective biomarkers for bladder cancer are eagerly expected in the related field.

Urine directly contacts the epithelial cells of bladder cancer. Therefore, urine may contain the proteins secreted by bladder cancer cells and regarded as the source for finding bladder cancer biomarkers. The most hopeful way to find useful bladder cancer biomarkers is to study the change of urine proteome during the progression of bladder cancer. The peptide spectral counts or emPAI (exponentially modified protein abundance index) can approximately estimate the contents of the defined proteins via the mass spectroscopy and database. However, the new analystic tactic of mass spectroscopy data is still rarely used in proteome analysis.

Accordingly, the present invention proposes a bladder cancer biomarker and a test method using the same to overcome the abovementioned problems.

### SUMMARY OF THE INVENTION

The primary objective of the present invention is to provide a bladder cancer biomarker and a test method using the same, which can assist in diagnosing bladder cancer and determining the aggressiveness of bladder cancer, whereby an optimized treatment can be used to promote the therapeutic effect.

Another objective of the present invention is to provide a bladder cancer biomarker and a test method using the same, which can cooperate with the existing methods, such as the occult blood test, NMP22 molecular test, cystoscopic examination and cytological examination, to improve the diagnosis of bladder cancer and the evaluation of the aggressiveness and malignancy of bladder cancer.

To achieve the abovementioned objectives, the present invention proposes a bladder cancer biomarker, which contains at least one of the following compounds: apolipoprotein A1 (APOA1), apolipoprotein A2 (APOA2), peroxiredoxin 2 (PRDX2), heparin cofactor 2 precursor (HCII), and serum amyloid A-4 protein (SAA4), and which exists in the urine of the testee.

The present invention also proposes a bladder cancer biomarker, which exists in the urine of the testee and contains at least one of the following 69 types of proteins: Protein S100-P (S100P), Cerulopasmin precursor (SP), Serum amyloid A-4 protein precursor (SAA4), Isoform 1 of Complement factor B precursor (Fragment)(CFB), Afamin precursor (AFM), Apolipoprotein A-I precursor (APOA1), Apolipoprotein A-II precursor (APOA2), Isoform 1 of Fibrinogen alpha chain precursor (FGA), Isoform Gamma-B of Fibrinogen alpha chain precursor (FGG), Apolipoprotein B-100 precursor (APOB), Alpha-1-acid glycolprotein 1 precursor (ORM1), Transthyretin precursor (TTR), ALB protein (ALB), Serotransferrin precursor (TF), Hemopexin precursor (HPX), Antithrombin III variant (SERPINC 1), Angiotensinogen precursor (AGT), 187 kDa protein (C3), FLJ00385 protein (Fragment)(IGHM), Glutathione S-transferase P (GSTP1), Fibrinogen beta chain precursor (FGB), Beta-2-glycoprotein 1 precursor (APOH), Complement C2 precursor (Fragment)(C2), Apolipoprotein A-IV precursor (APOA4), ENO1P protein (EDARAD), Hemoglobin subunit alpha (HBA1; HBA2), Peptidyl-prolyl cis-transisomerase A (PPlA; LOC 654188; LOC 653214), Hemoglobin subunit delta (HBD; HBB), Alpha-2-macroglobulin precursor (A2M), Alpha-1-antitrypsin precursor (SERPINA1), Vitamin D-binding protein precursor (GC), Immunglobulin heavy chain variable region, Myosin-reactive immunoglobulin heavy chain variable region (Fragment), Heparin cofactor 2 precursor (SERPIND1 (HCII)), Peroxiredoxin-2 (PRDX2), heterogeneous nuclear ribonucleoprotein D-like (HNRPDL), Keratin-8-like protein 1, Isoform 2 of Apolipoprotein-L1 precursor (APOL1), Ig heavy chain V-I region EU, Protein S100-A6 (S100A6), Fetuin-B precursor (FETUB), Factor VII active site mutant immunoconjugate (F7), RcTPI1 (Fragment) (LOC729708), Isoform a 1 of Acyl-CoA-binding protein (DBI), IGHA1 protein (IGHA1), Ig heavy chain V-III region GAL, Macrophage migration inhibitory factor (MIF), 14-3-3 protein theta (YWHAQ), Ig mu heavy chain disease protein, HP protein (HP), Serum paraoxonase/arylesterase 1 (PON1), Complement component C9 precursor (C9), Fructose-bisphosphate aldolase A (ALDOA), Kallikrein B, plasma (Fletcher factor) 1 (KLKB1), Inter-alpha-trypsin inhibitor heavy chain H2 precursor (ITIH2), Protein S100-A4 (S100A4), Malate dehydrogenase, mitochondrial precursor (MDH2), Protein S100-A11 (S100A11), Extracellular matrix protein 1 precursor (ECM1), Complement factor H-related protein 3 precursor (CFHR3), Hemoglobin subunit beta (HBB), 101 kDa protein (NDST1), Nucleoside diphosphate kinase A (NME1), Apolipoprotein C-III precursor (APOC3), Histone H4 (HIST1H4), Isoform 1 of Haptoglobin-related protein precursor (HPR), TALDO1 protein (TALDO1), IGHG4 protein (IGHG4), and Myosin-reactive immunoglobulin heavy chain variable region (Fragment).

The present invention also proposes a method for inspecting bladder cancer, which comprises steps: providing a urine specimen of a testee; providing at least one biomarker; and detecting the expression intensity of the biomarker in the urine specimen, wherein the biomarker contains at least one selected from the abovementioned 69 types of proteins or selected from a group consisting of apolipoprotein A1 (APOA1), apolipoprotein A2 (APOA2), peroxiredoxin 2 (PRDX2), heparin cofactor 2 precursor (HCII), and serum amyloid A-4 protein (SAA4).

Below, the embodiments are described in detail to make easily understood the objectives, technical contents, characteristics and accomplishments of the present invention.

### BRIEF DESCRIPTION OF THE DRAWINGS

FIG. 1 is a flowchart of a method for detecting bladder cancer according to one embodiment of the present invention; and

FIG. 2 contains a group of diagrams FIGS. 2(a)-2(e) schematically showing detection of five candidate proteins in individual urine proteins by a western blot assay (lower panel), wherein the fold changes between NT, LgEs, HgEs, HsAs and UTI/HU are calculated according to the constant total urine protein amounts (the left panel) and the equal volume of urine (the right panel).

### DETAILED DESCRIPTION OF THE INVENTION

The present invention discloses 69 types of urine proteins (listed in Table. 1) specific to bladder cancer as new biomarkers. When the 69 proteins are highly expressed in the urine of a patient, it means that the patient is more likely to have bladder cancer or that the patient is more seriously invaded by bladder cancer cells. Among the 69 bladder cancer-specific proteins, more attention is paid to the following five proteins: apolipoprotein A1 (APOA1), apolipoprotein A2 (APOA2), peroxiredoxin 2 (PRDX2), heparin cofactor 2 precursor (HCII), and serum amyloid A-4 protein (SAA4).

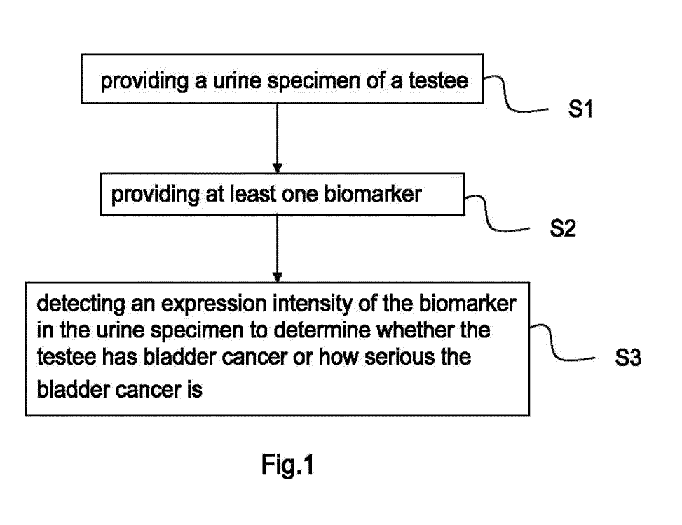

Refer to FIG. 1 a flowchart of a method for detecting bladder cancer according to one embodiment of the present invention. In Step S1, provide a urine specimen of a testee. In Step S2, provide at least one biomarker, which is selected from the abovementioned 69 bladder cancer-specific proteins. In Step S3, detect the expression intensity of the biomarker in the urine specimen to determine whether the testee has bladder cancer or how serious the bladder cancer is.

The expression intensity of the biomarker in the urine specimen of the testee is compared with the expression intensity of the biomarker in the urine specimens of healthy persons or the former urine specimen of the same testee to determine whether the testee has bladder cancer or determine how seriously the bladder cancer invades.

The method of the present invention can cooperate with the existing methods, such as the occult blood test, NMP22 molecular test, cystoscopic examination and cytological examination, to improve the diagnosis of bladder cancer and the evaluation of the aggressiveness and malignancy of bladder cancer.

The present invention uses an iTRAQ (Isobaric Tagging for Relative and Absolute Quantification) analysis technology to find out the proteins whose concentrations are abnormal in urine. The effect of the present invention is further verified with the western blot method in individual samples. In the present invention, a marker panel, which contains one or more proteins respectively having abnormal expressions, is used to implement the early detection, diagnosis and staging of bladder cancer. Thereby, the physician can arrange an optimized treatment to achieve the best therapeutic effect.

The contents of the biomarkers are verified with a western blot method in the embodiments of the present invention. However, the mass spectrometric method, fluorescent method, luminescent method, immunological method, chromatographic method, etc., may also apply to detecting the contents of the biomarkers.

### Embodiments

### iTRAQ Labeling and Fractionation by Strong Cationic Exchange (SCX) and Basic Reverse Phase (RP) Chromatography

In an experiment, the urine proteins of the patients, who meet the age control condition and are at the same histological stage or the same pathological stage, are mixed to decrease individual differences and enhance the signals. In this experiment, hernial patients are selected to form a sub-group of the non-tumor (NT) control group. The mixed urine proteins (100 μg) of each sub-group are processed according to the operational proposal of the manufacturer of a four-plex iTARQ (Applied Biosystems, Foster City, Calif.). In this experiment, 100 μg protein is labeled with one unit of iTRAQ reagent. One unit of iTARQ agent is dissolved in 70 μL alcohol. The alcohol solution is added to the mixed urine proteins of each subgroup to enable reduction, cysteine-blocking and trypsin hydrolysis. The iTARQ agents of 114, 115, 116 and 117 tags reagents are respectively added to the peptide fragments of the NT control group, the low-grade/early-stage (LgEs) sub-group, and the high-grade/advanced-stage (HgAs) sub-group to undertake reactions at an ambient temperature for one hour. Then, the four groups of samples are mixed and dried with a vacuum centrifugal method. Then, the clinical sample set 1 is fractioned by SCX chromatography. The clinical sample set 2 is fractioned by SCX chromatography and RP chromatography respectively. The elution fractions are pooled as 42 fractions and vacuum dried for nano ESI-LC-MS/MS analysis.

### LC-ESI MS/MS Analysis by LTQ-Orbitrap PQD

Each separated peptide fraction is reconstituted in buffer E (0.1% formic acid in H₂O). 2 μg peptides of each fraction were loaded across a trap column (Zorbax 300SB-C18, 0.3×5 mm, Agilent Technologies, Wilmington, Del., USA) at a flow rate of 0.2 μL/min in buffer A, and separated on a resolving 10-cm analytical BioBasic® C₁₈ PicoFrit™ column (inner diameter, 75 μm) with a 15 μm tip (New Objective, Woburn, Mass.). The LC setup is coupled on line to a linear ion trap-orbitrap (LTQ-Orbitrap) (Thermo Fisher, San Jose, Calif., USA) operated by the Xcalibur 2.0 software (Thermo Fisher). Peptide fragments are detected in LTQ for MS/MS in a pulsed Q dissociation (PQD) operating mode with a normalized collision energy setting of 27%. One MS scan is followed by three MS/MS scans. The m/z scan range for MS scans is 350 to 2000 Da.

### Protein Identification and Quantification by Sequence Database Searching

For protein identification, a MASCOT engine (Matrix Science, London, UK; version 2.2.04) is used to search the resulting MS/MS spectra against a non-redundant International Protein Index (IPI) human sequence database v3.27 (released at March 2007; 67,528 sequences; 28,353,548 residues) from the European Bioinformatics Institute (http://www.ebi.ac.uk/). 10 ppm mass tolerance is permitted for intact peptide masses and 0.5 Da for PQD fragment ions. Two missed cleavages made from the trypsin digest are allowed. Oxidized methionine is set as a potential variable modification, and iTRAQ (N terminal), iTRAQ (K), and MMTS (C) are set as the fixed modifications. The charge states of peptides are set as +2 and +3. The validation of protein identification and quantification are performed with the open source Trans-Proteomic Pipeline (TPP) software (Version 4.0). The MASCOT search results in the DAT file for each SCX or RP elution. The MS raw data and the DAT files containing information of identified peptides are then processed and analyzed in the TPP software. In the present invention, both the PeptideProphet and ProteinProphet probability scores >0.95 are used to ensure an overall false positive rate below 0.7%. Quantification of the ratio of each protein was achieved with the Libra program. A module within the TPP is used to perform quantification on MS/MS spectra. The minimum intensity threshold of reporter ion is 20.

### Western Blot Analysis

After desalting and concentration of urine proteins, the protein amount of each urine sample is measured by the DC protein assay. The western blot method is used to analyze the samples of urine. Urine protein (100 μg) from individual or pooled sample is resolved in SDS-gels and transferred electrophoretically onto a PVDF membrane (Bio-Rad, Hercules Calif., USA). The membrane is blocked with 5% non-fat dried milk in trisbuffered saline (Bio-Rad, Hercules Calif., USA) with 0.1% Tween-20 (Sigma, St Louis) (TBST) for 1 h at an ambient temperature. The following antibodies are used in the western blot analysis: anti-apolipoprotein A-I (anti-APOA1, 1:500, ab58924, Abcam), anti-apolipoprotein A-II (anti-APOA2, 1:250, ab54796, Abcam), anti-heparin cofactor 2 precursor (anti-HC2, 1:2000, MAB0769, Abnova), anti-peroxiredoxin 2 (anit-PRDX2, 1:5000, AF3489, R&D systems), anti-s100A6 (1:200, AF4584, R&D systems), and anti-s100A8 (1:200, AF4570, R&D systems). In the west blot analysis, the membranes were probed with primary antibody followed by horseradish peroxidase-conjugated antibody and developed with enhanced chemiluminescence detection according to the manufacturer's instructions (Millpore, Mass., USA). To quantify the urine APOA1 level absolutely, apolipoprotein A-I protein purified from human plasma (A0722, Sigma-Aldrich, USA) is used as a quantitative standard in western blot analyses.

### Statistical Analysis

To perform the statistical analysis of the western blotting results, the individual results are statistically analyzed with means, standard deviations, medians, and interquartile ranges. The differences in urine concentrations of APOA1, APOA2, PRDX2 and HCII in the control group and cancer patients are measured with the nonparametric Mann-Whitney U test. Statistical analyses are conducted with the SPSS software (version 13.0, SPSS Inc, Chicago, Ill.). Two-tailed p values of 0.05 or less are considered significant. Receiver operator characteristic (ROC) curve analysis and the area under the curve (AUC) are also calculated.

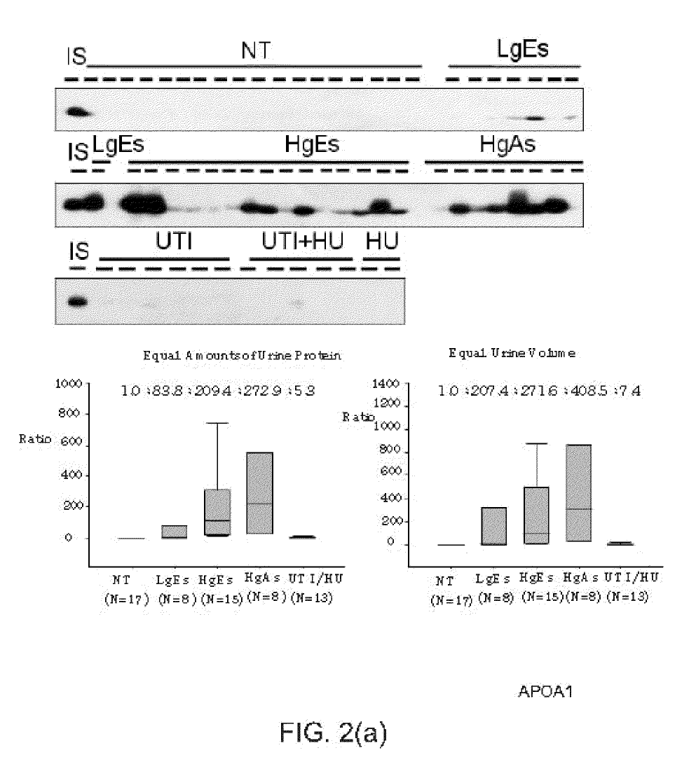

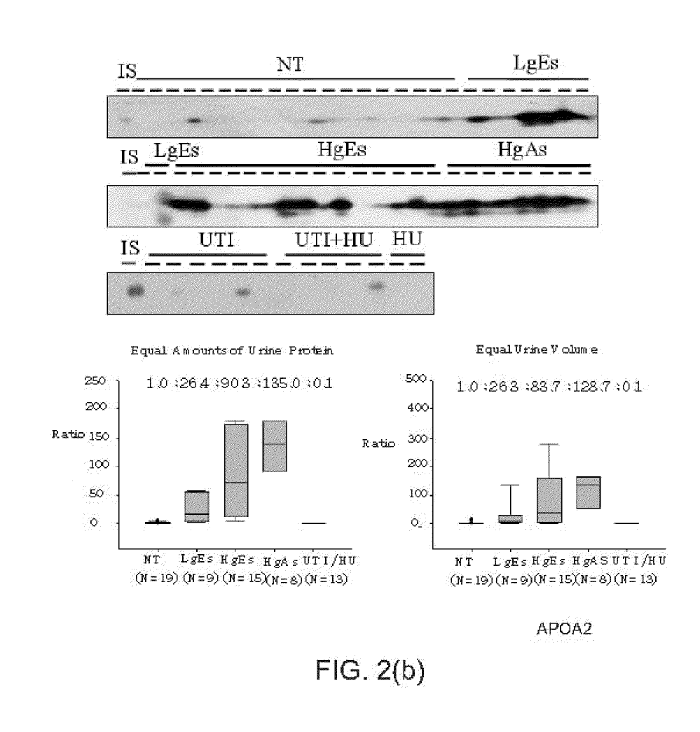

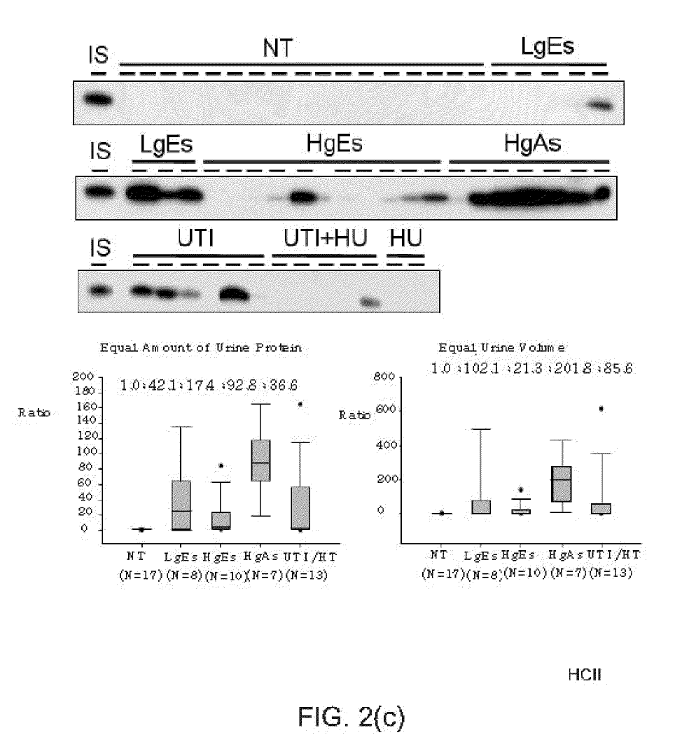

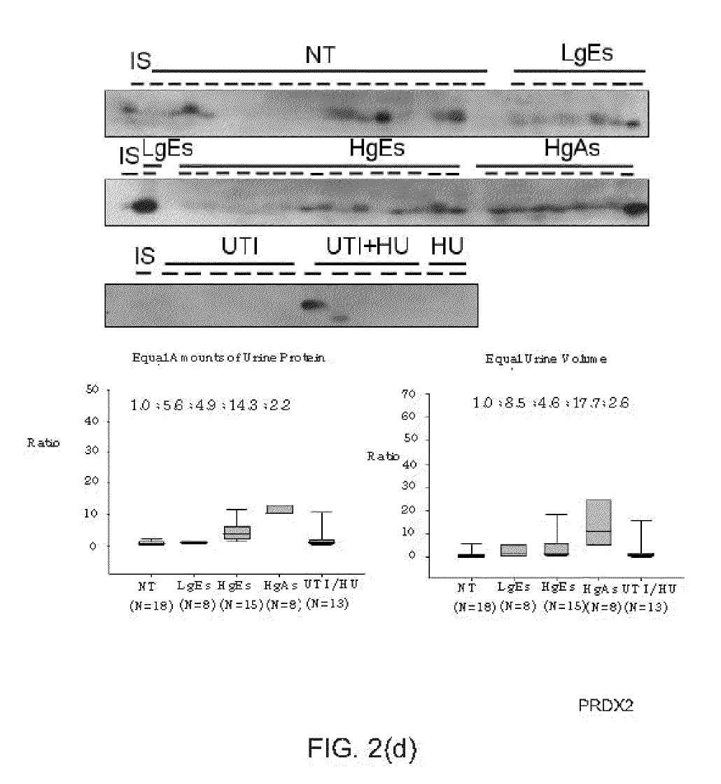

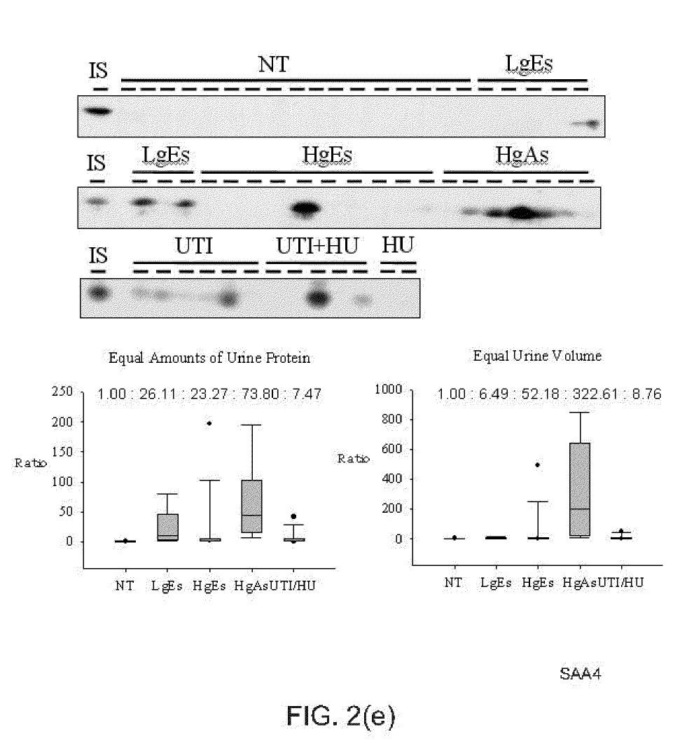

Refer to FIG. 2 a group of diagrams schematically showing the detection of five candidate proteins in individual urine proteins by a western blotting assay (the lower panel). Equal amounts of proteins prepared from individual urine samples are separated by SDS-PAGE, transferred to PVDF membranes, and probed with the associated antibodies. FIGS. 2(a)-2(e) respectively show the detection results:(a) APOA1 in 100 μg urine protein, (b) APOA2 in 100 μg urine protein, (c) HCII in 50 μg urine protein, (d) PRDX2 in 100 μg urine protein, and (e) SAA4 in 50 μg urine protein. The fold changes between NT, LgEs, HgEs, HsAs and UTI/HU are calculated according to the constant total urine protein amounts (the left panel) and the equal volume urine (the right panel). The average fold change of every subgroup by comparing with NT subgroups is labeled on the top of each box plot. The horizontal lines of the box plot in each lane indicate the 10^{th}, 25^{th}, 50^{th}, 75^{th} and 90^{th} percentiles of data points.

Table. 1 Lists iTRAQ ratios of the 69 up-expressed proteins in urine samples of bladder cancer patients

| Accession | | Gene | Clinical sample set 1 | | | |
|---|---|---|---|---|---|---|
| Number | Protein Name | Name | NT ± SD | LgEs/NT ± SD | HgEs/NT ± SD | HgAs/NT ± SD |
| IPI00017526.1 | Protein S100-P | S100P | 1 | 1.42 | 50.83 | 29.27 |
| IPI00017601.1 | Ceruloplasmin | CP | 1 ± 0.01 | 1.01 ± 0.01 | 6.86 ± 0.01 | 6.54 ± 0.01 |
| | precursor | | | | | |
| IPI00019399.1 | Serum amyloid A-4 | SAA4 | 1 ± 0.04 | 1.43 ± 0.05 | 15.77 ± 0.06 | 15.41 ± 0.12 |
| | protein precursor | | | | | |
| IPI00019591.1 | Isoform 1 of | CFB | 1 ± 0.04 | 0.78 ± 0.03 | 5.98 ± 0.03 | 6.7 ± 0.04 |
| | Complement factor B | | | | | |
| | precursor (Fragment) | | | | | |
| IPI00019943.1 | Afamin precursor | AFM | 1 ± 0.04 | 1.18 ± 0.06 | 5.07 ± 0.05 | 7.12 ± 0.06 |
| IPI00021841.1 | Apolipoprotein A-I | APOA1 | 1 ± 0 | 2.01 ± 0.01 | 32.25 ± 0.02 | 41.66 ± 0.02 |
| | precursor | | | | | |
| IPI00021854.1 | Apolipoprotein A-II | APOA2 | 1 ± 0 | 2.42 ± 0 | 71.87 ± 0.05 | 124.9 ± 0.06 |
| | precursor | | | | | |
| IPI00021885.1 | Isoform 1 of | FGA | 1 ± 0.01 | 2.85 ± 0.01 | 16.92 ± 0.02 | 35.71 ± 0.03 |
| | Fibrinogen alpha | | | | | |
| | chain precursor | | | | | |
| IPI00021891.5 | Isoform Gamma-B of | FGG | 1 ± 0.01 | 1.56 ± 0.01 | 11.09 ± 0.02 | 29.1 ± 0.02 |
| | Fibrinogen gamma | | | | | |
| | chain precursor | | | | | |
| IPI00022229.1 | Apolipoprotein B-100 | APOB | 1 | 0.93 | 3.1 | 9.92 |
| | precursor | | | | | |
| 1PI00022429.3 | Alpha-1-acid | ORM1 | 1 ± 0.02 | 2.46 ± 0.04 | 4.78 ± 0.03 | 2.22 ± 0.03 |
| | glycoprotein 1 | | | | | |
| | precursor | | | | | |
| IPI00022432.1 | Transthyretin | TTR | 1 ± 0.04 | 1.26 ± 0.03 | 3.28 ± 0.03 | 3.95 ± 0.04 |
| | precursor | | | | | |
| IPI00022434.2 | ALB protein | ALB | 1 ± 0 | 1.37 ± 0 | 11.81 ± 0 | 15.94 ± 0.01 |
| IPI00022463.1 | Serotransferrin | TF | 1 ± 0 | 1.38 ± 0 | 21.01 ± 0.01 | 21.71 ± 0.01 |
| | precursor | | | | | |
| IPI00022488.1 | Hemopexin precursor | HPX | 1 ± 0.01 | 0.84 ± 0.01 | 10.01 ± 0.01 | 16.86 ± 0.02 |
| IPI00032179.2 | Antithrombin III | SERPIN C1 | 1 ± 0.01 | 1.12 ± 0.01 | 7.25 ± 0.03 | 4.25 ± 0.03 |
| | variant | | | | | |
| IPI00032220.3 | Angiotensinogen | AGT | 1 ± 0 | 0.61 ± 0 | 18.25 ± 0.01 | 31.95 ± 0.01 |
| | precursor | | | | | |
| IPI00164623.4 | 187 kDa protein | C3 | 1 ± 0.01 | 1.77 ± 0.01 | 19.79 ± 0.01 | 27.84 ± 0.01 |
| IPI00168728.1 | FLJ00385 protein | IGHM | 1 ± 0.04 | 0.73 ± 0.04 | 3.53 ± 0.04 | 8.54 ± 0.09 |
| | (Fragment) | | | | | |
| IPI00219757.13 | Glutathione | GSTP1 | 1 ± 0.01 | 1.58 ± 0.01 | 9.13 ± 0.01 | 5.83 ± 0.01 |
| | S-transferase P | | | | | |
| IPI00298497.3 | Fibrinogen beta chain | FGB | 1 ± 0.01 | 5.24 ± 0.01 | 8.63 ± 0.01 | 45.36 ± 0.01 |
| | precursor | | | | | |
| IPI00298828.3 | Beta-2-glycoprotein 1 | APOH | 1± 0 | 1.18 ± 0.01 | 6.68 ± 0.01 | 13.68 ± 0.02 |
| | precursor | | | | | |
| IPI00303963.1 | Complement C2 | C2 | 1 ± | 1.16 | 5.07 | 6.06 |
| | precursor (Fragment) | | | | | |
| IPI00304273.2 | Apolipoprotein A-IV | APOA4 | 1 ± 0.01 | 1.14 ± 0.01 | 5.19 ± 0.01 | 10.17 ± 0.01 |
| | precursor | | | | | |
| IPI00328587.4 | ENO1P protein | EDARAD D | 1 ± 0.02 | 0.94 ± 0.01 | 5.22 ± 0.02 | 2.73 ± 0.02 |
| IPI00410714.5 | Hemoglobin subunit | HBA1; HBA2 | 1 ± 0.01 | 0.89 ± 0.01 | 16.76 ± 0.01 | 65.81 ± 0.01 |
| | alpha | PPIA; LO | | | | |
| IPI00419585.9 | Peptidyl-prolyl | C654188; | 1 ± 0.01 | 0.84 ± 0.01 | 11.64 ± 0.01 | 6.33 ± 0 |
| | cis-trans isomerase A | LOC653214 | | | | |
| IPI00473011.3 | Hemoglobin subunit | HBD; HBB | 1 ± 0.01 | 0.81 ± 0.01 | 11.32 ± 0.02 | 30.75 ± 0.01 |
| | delta | | | | | |
| IPI00478003.1 | Alpha-2-macroglobulin | A2M | 1 ± 0.01 | 1.3 ± 0.01 | 14.02 ± 0.01 | 20.94 ± 0.01 |
| | precursor | | | | | |
| IPI00553177.1 | Alpha-1-antitrypsin | SERPIN A1 | 1 ± 0 | 1.22 ± 0 | 12.45 ± 0.01 | 11.21 ± 0.01 |
| | precursor | | | | | |
| IP100555812.4 | Vitamin D-binding | GC | 1 ± 0.01 | 1.82 ± 0.01 | 15.57 ± 0.02 | 25.32 ± 0.03 |
| | protein precursor | | | | | |
| IPI00745363.3 | Immunglobulin heavy | — | 1+0.01 | 1.17 ± 0.02 | 4.54 ± 0.03 | 7.06 ± 0.01 |
| | chain variable region | | | | | |
| IPI00783024.1 | Myosin-reactive | — | 1 | 0.93 | 6.42 | 14.49 |
| | immunoglobulin | | | | | |
| | heavy chain variable | | | | | |
| | region (Fragment) | | | | | |
| IPI00292950 | Heparin cofactor 2 | SERPIN D1 | | | | |
| | precursor | (HCII) | | | | |
| IPI00027350 | Peroxiredoxin-2 | PRDX2 | | | | |
| IPI00011274 | heterogeneous nuclear | HNRPDL | 1 | 2.87 | 16.37 | 8.64 |
| | ribonucleoprotein | | | | | |
| | D-like | | | | | |
| IPI00017870 | Keratin-8-like protein 1 | — | 1 | 2.33 | 27.17 | 14.89 |
| IPI00186903 | Isoform 2 of | APOL1 | 1 | 1.53 | 25.74 | 21.61 |
| | Apolipoprotein-L1 | | | | | |
| | precursor | | | | | |
| IPI00382455 | Ig heavy chain V-I | — | 1 | 1.52 | 11.85 | 23.71 |
| | region EU | | | | | |
| IPI00027463 | Protein S100-A6 | S100A6 | 1 | 1.36 | 7.93 | 4.87 |
| IPI00005439 | Fetuin-B precursor | FETUB | 1 | 1.15 | 5.21 | 5.01 |
| IPI00382606 | Factor VII active site | F7 | 1 | 1.04 | 4.06 | 5.85 |
| | mutant | | | | | |
| | immunoconjugate | | | | | |
| IPI00383071 | RcTPI1 (Fragment) | LOC729708 | 1 | 1.01 | 5.74 | 2.59 |
| IPI00010182 | Isoform a 1 of | DBI | 1 | 0.95 | 6.16 | 5.13 |
| | Acyl-CoA-binding | | | | | |
| | protein | | | | | |
| IPI00166866 | IGHA1 protein | IGHA1 | 1 | 0.94 | 6.73 | 10.78 |
| IPI00382500 | Ig heavy chain V-III | — | 1 | 0.87 | 6.2 | 6.16 |
| | region GAL | | | | | |
| IPI00293276 | Macrophage migration | MIF | 1 | 0.83 | 5.92 | 3.98 |
| | inhibitory factor | | | | | |
| IPI00018146 | 14-3-3 protein theta | YWHAQ | 1 | 0.83 | 5.05 | 2.73 |
| IPI00385264 | Ig mu heavy chain | — | 1 | 0.61 | 6.92 | 8.52 |
| | disease protein | | | | | |
| IPI00431645 | HP protein | HP | 1 | 0.59 | 27.71 | 14.81 |
| IPI00218732 | Serum paraoxonase/ | PON1 | | | | |
| | arylesterase 1 | | | | | |
| IPI00022395 | Complement component C9 | C9 | | | | |
| | precursor | | | | | |
| IPI00465439 | Fructose-bisphosphate | ALDOA | | | | |
| | aldolase A | | | | | |
| IPI00654888 | Kallikrein B, plasma | KLKB1 | | | | |
| | (Fletcher factor) 1 | | | | | |
| IPI00305461 | Inter-alpha-trypsin | ITIH2 | | | | |
| | inhibitor heavy chain | | | | | |
| | H2 precursor | | | | | |
| IPI00032313 | Protein S100-A4 | S100A4 | | | | |
| IPI00291006 | Malate dehydrogenase, | MDH2 | | | | |
| | mitochondrial | | | | | |
| | precursor | | | | | |
| IPI00013895 | Protein S100-A11 | S100A11 | | | | |
| IPI00003351 | Extracellular matrix | ECM1 | | | | |
| | protein 1 precursor | | | | | |
| IPI00027507 | Complement factor | CFHR3 | | | | |
| | H-related protein 3 | | | | | |
| | precursor | | | | | |
| IPI00654755 | Hemoglobin subunit beta | HBB | | | | |
| IP100005599 | 101 kDa protein | NDST1 | | | | |
| IPI00012048 | Nucleoside | NME1 | | | | |
| | diphosphate kinase A | | | | | |
| 1PI00021857 | Apolipoprotein C-III | APOC3 | | | | |
| | precursor | | | | | |
| IPI00453473 | Histone H4 | HIST1H4 | | | | |
| IP100477597 | Isoform 1 of | HPR | | | | |
| | Haptoglobin-related | | | | | |
| | protein precursor | | | | | |
| IPI00550488 | TALDO1 protein | TALDO1 | | | | |
| IPI00550640 | IGHG4 protein | IGHG4 | | | | |
| IPI00786926 | Myosin-reactive | — | | | | |
| | immunoglobulin | | | | | |
| | heavy chain variable | | | | | |
| | region (Fragment) | | | | | |

| Accession | Clinical sample set 2 | | | |
|---|---|---|---|---|
| Number | NT ± SD | LgEs/NT ± SD | HgEs/NT ± SD | HgAs/NT ± SD |
| IPI00017526.1 | 1 ± 0.01 | 4.84 ± 0.01 | 1.5 ± 0.01 | 1.89 ± 0.01 |
| IPI00017601.1 | 1 ± 0 | 2.55 ± 0.01 | 1.84 ± 0 | 3.03 ± 0.01 |
| IPI00019399.1 | 1 ± 0.02 | 3.9 ± 0.02 | 2.77 ± 0.01 | 5.31 ± 0.01 |
| IPI00019591.1 | 1 ± 0.05 | 4.04 ± 0.04 | 2.07 ± 0.04 | 6.69 ± 0.04 |
| IPI00019943.1 | 1 ± 0.01 | 3.95 ± 0.01 | 3.58 ± 0.01 | 5.58 ± 0.01 |
| IPI00021841.1 | 1 ± 0.01 | 14.39 ± 0.01 | 4.52 ± 0.01 | 18.12 ± 0.01 |
| IPI00021854.1 | 1 ± 0.02 | 2.21 ± 0.03 | 1.27 ± 0.01 | 2.62 ± 0.02 |
| IPI00021885.1 | 1 ± 0.01 | 2.8 ± 0.01 | 2.39 ± 0.01 | 12.85 ± 0.01 |
| IPI00021891.5 | 1 ± 0.01 | 9.02 ± 0.01 | 2 ± 0 | 30.93 ± 0.01 |
| IPI00022229.1 | 1 ± 0.01 | 9.4 ± 0.01 | 2.55 ± 0.01 | 7.3 ± 0.02 |
| 1PI00022429.3 | 1 ± 0.01 | 1.45 ± 0.01 | 3.23 ± 0.01 | 1.46 ± 0.01 |
| IPI00022432.1 | 1 ± 0.01 | 4.01 ± 0.02 | 2.25 ± 0.01 | 6.26 ± 0.02 |
| IPI00022434.2 | 1 ± 0 | 5.13 ± 0 | 3.69 ± 0 | 7.5 ± 0 |
| IPI00022463.1 | 1 ± 0 | 5.36 ± 0 | 3.6 ± 0 | 7.17 ± 0 |
| IPI00022488.1 | 1 ± 0.01 | 2.78 ± 0.01 | 2.04 ± 0.01 | 4.36 ± 0.01 |
| IPI00032179.2 | 1 ± 0 | 3.29 ± 0.01 | 2.57 ± 0 | 4.35 ± 0.01 |
| IPI00032220.3 | 1 ± 0.01 | 1.88 ± 0.01 | 2.48 ± 0.01 | 2.25 ± 0.02 |
| IPI00164623.4 | 1 ± 0 | 10.33 ± 0 | 3.37 ± 0 | 13.81 ± 0.01 |
| IPI00168728.1 | 1 ± 0.01 | 2.37 ± 0.01 | 1.07 ± 0.01 | 3.35 ± 0.01 |
| IPI00219757.13 | 1 | 2.81 | 0.88 | 2.68 |
| IPI00298497.3 | 1 ± 0 | 15.07 ± 0.01 | 3.16 ± 0 | 54 ± 0.01 |
| IPI00298828.3 | 1 ± 0.03 | 3.05 ± 0.02 | 1.93 ± 0.02 | 3.56 ± 0.03 |
| IPI00303963.1 | 1 | 2.06 | 1.23 | 3.22 |
| IPI00304273.2 | 1 ± 0.01 | 3.56 ± 0.01 | 3.58 ± 0.01 | 3.48 ± 0.01 |
| IPI00328587.4 | 1 ± 0.01 | 1.32 ± 0.01 | 1.13 ± 0.01 | 7.16 ± 0.03 |
| IPI00410714.5 | 1 ± 0 | 4.29 ± 0.01 | 3.07 ± 0.01 | 100.37 ± 0.01 |
| IPI00419585.9 | 1 ± 0.01 | 1.27 ± 0.01 | 1.02 ± 0.01 | 2.26 ± 0.01 |
| IPI00473011.3 | 1 ± 0.01 | 4.01 ± 0.01 | 2.51 ± 0.02 | 49.05 ± 0.03 |
| IPI00478003.1 | 1 ± 0.01 | 10.17 ± 0.01 | 4.75 ± 0 | 16.6 ± 0.01 |
| IPI00553177.1 | 1 ± 0 | 3.91 ± 0 | 3.31 ± 0 | 5.98 ± 0 |
| IP100555812.4 | 1 ± 0 | 5.79 ± 0.01 | 3.29 ± 0 | 8.87 ± 0.01 |
| IPI00745363.3 | 1 ± 0 | 2.35 ± 0.01 | 1.47 ± 0.02 | 3.6 ± 0.01 |
| IPI00783024.1 | 1 ± 0.02 | 1.97 ± 0.02 | 1.25 ± 0.02 | 3.34 ± 0.02 |
| IPI00292950 | 1 | 6.31 | 3.26 | 5.75 |
| IPI00027350 | 1 | 2.27 | 1.54 | 12.15 |
| IPI00011274 | | | | |
| IPI00017870 | | | | |
| IPI00186903 | | | | |
| IPI00382455 | | | | |
| IPI00027463 | | | | |
| IPI00005439 | | | | |
| IPI00382606 | | | | |
| IPI00383071 | | | | |
| IPI00010182 | | | | |
| IPI00166866 | | | | |
| IPI00382500 | | | | |
| IPI00293276 | | | | |
| IPI00018146 | | | | |
| IPI00385264 | | | | |
| IPI00431645 | | | | |
| IPI00218732 | 1 | 12.3 | 3.4 | 14.6 |
| IPI00022395 | 1 | 2.18 | 2.5 | 4.5 |
| IPI00465439 | 1 | 3.11 | 1.3 | 6.93 |
| IPI00654888 | 1 | 11.8 | 6.6 | 24.8 |
| IPI00305461 | 1 | 18.9 | 5.1 | 20.7 |
| IPI00032313 | 1 | 7.56 | 2.2 | 5.39 |
| IPI00291006 | 1 | 2.52 | 1.7 | 4.81 |
| IPI00013895 | 1 | 4.96 | 2.4 | 4.83 |
| IPI00003351 | 1 | 2.94 | 1.4 | 4.1 |
| IPI00027507 | 1 | 9.28 | 4.2 | 9.43 |
| IPI00654755 | 1 | 3.27 | 1.7 | 32.9 |
| IP100005599 | 1 | 2.98 | 2.2 | 5.24 |
| IPI00012048 | 1 | 2.34 | 1.3 | 5.28 |
| 1PI00021857 | 1 | 1.84 | 0.9 | 7.3 |
| IPI00453473 | 1 | 4.33 | 4.1 | 3.05 |
| IP100477597 | 1 | 5.77 | 5.2 | 20.7 |
| IPI00550488 | 1 | 2.21 | 1.3 | 5.64 |
| IPI00550640 | 1 | 8.26 | 3.1 | 7.21 |
| IPI00786926 | 1 | 2.7 | 2.9 | 6.08 |

Table. 2 shows the average fold changes of the selected urine proteins in the LgEs, HgEs, HgAs and UTI/Hematuria subgroups compared to the NT group

TABLE 2

| | NT (N = | LgEs (N = 8 or | HgEs (N = 10 | | UT/Hematuria |
|---|---|---|---|---|---|
| | 17-19) | 9) | or 15) | HgAs (N = 7 or 8 | (N = 13) |
| Constant total | | | | | |
| urine protein | | | | | |
| APOA1 | 1 ± 0.4 | 83.8 ± 186.5 | 209.4 ± 252.9 | 272.9 ± 252.1 | 5.3 ± 4.4 |
| APOA1 | 0.1 ± 0.2 | 1.2 ± 1.8 | 2.1 ± 2.6 | 3.2 ± 3.5 | 0.7 ± 0.8 |
| (ng/μg urine | (N = 37) | (N = 14) | (N = 24) | (N = 11) | (N = 18) |
| protein) | | | | | |
| APOA2 | 1 ± 1.3 | 26.4 ± 23.8 | 90.3 ± 75.1 | 135.0 ± 47.9 | 0.1 ± 0.1 |
| HCII | 1 ± 0.3 | 42.1 ± 55.5 | 17.4 ± 26.8 | 92.8 ± 52.3 | 36.3 ± 55.2 |
| PRDX2 | 1 ± 0.9 | 5.6 ± 13.5 | 4.9 ± 3.5 | 14.3 ± 10.5 | 2.2 ± 4.2 |
| SAA4 | 1 | 26.11 | 23.27 | 73.80 | 7.47 |
| Constant urine | | | | | |
| volume | | | | | |
| APOA1 | 1 ± 1.4 | 207.4 ± 406.0 | 271.6 ± 338.0 | 408.5 ± 408.2 | 7.4 ± 8.9 |
| APOA1 | 5.0 ± 14.2 | 624.1 ± 1433.9 | 1631.0 ± 7362.0 | 920.9 ± 1347.3 | 269.7 ± 532.3 |
| (ng/mL | (N = 50) | (N = 21) | (N = 39) | (N = 16) | (N = 25) |
| urine) | | | | | |
| APOA2 | 1 ± 2.7 | 26.3 ± 42.1 | 83.7 ± 110.3 | 128.7 ± 81.8 | 0.1 ± 0.2 |
| HCII | 1 ± 1.2 | 102.1 ± 225.4 | 21.3 ± 42.8 | 201.8 ± 155.7 | 85.6 ± 192.8 |
| PRDX2 | 1 ± 2.0 | 8.5 ± 19.2 | 4.6 ± 6.0 | 17.7 ± 21.1 | 2.6 ± 6.7 |
| SAA4 | 1 | 6.49 | 52.18 | 322.61 | 8.76 |

Table. 3(a) shows summary of the p values, AUC values of the ROC curves, sensitivity and specificity of APOA1, APOA2, HC2, and PRDX2 expression by western blotting in constant amounts of total urine protein of individual samples.

TABLE 3(a)

| | p value | | p value of | | p value of | | | |
|---|---|---|---|---|---|---|---|---|
| | of | | early | | grade | | p value of stage | |
| | diagnosis | AUC | detection | AUC | differentiation | AUC | differentiation | AUC |
| protein | (controls: | (sensivity/ | (control: | (sensivity/ | LgEs: | (sensivity/ | (LgEs + HgEs): | (sensivity/ |
| name | all BC) | specificity) | LgEs) | soecificity) | (HgEs + HgAs) | soecificity) | HgAs | soecificity) |
| APOA1 | <0.001 | 0.972 | 0.002 | 0.890 | 0.010 | 0.810 | 0.299 | 0.625 |
| APOA2 | <0.001 | 0.987 | <0.001 | 0.971 | 0.004 | 0.831 | 0.012 | 0.802 |
| HCII | <0.001 | 0.885 | 0.221 | 0.654 | 0.322 | 0.625 | 0.003 | 0.889 |
| PRDX2 | <0.001 | 0.869 | 0.317 | 0.625 | 0.002 | 0.870 | 0.001 | 0.897 |
| SAA4 | <0.001 | 1 | 0.01 | 0.974 | 0.5 | 0.515 | 0.06 | 0.833 |

Table. 3(b) shows Summary of the p values, AUC values of the ROC curves, sensitivity and specificity of APOA1, APOA2, HC2, and PRDX2 expression by western blotting in constant urine volume of individual samples.

TABLE 3(b)

| | | | p value of | | p value of | | | |
|---|---|---|---|---|---|---|---|---|
| | p value of | | early | | grade | | p value of stage | |
| | diagnosis | AUC | detection | AUC | differentiation | AUC | differentiation | AUC |
| protein | (controls: all | (sensivity/ | (control: | (sensivity/ | LgEs: | (sensivity/ | (LgEs + HgEs): | (sensivity/ |
| name | BC) | specificity) | LgEs) | soecificity) | (HgEs + HgAs) | soecificity) | HgAs | soecificity) |
| APOA1 | <0.001 | 0.960 | 0.004 | 0.868 | 0.078 | 0.712 | 0.223 | 0.647 |
| APOA2 | <0.001 | 0.970 | <0.001 | 0.947 | 0.062 | 0.715 | 0.019 | 0.781 |
| HCII | <0.001 | 0.840 | 0.103 | 0.706 | 0.268 | 0.640 | 0.005 | 0.865 |
| PRDX2 | <0.001 | 0.851 | 0.059 | 0.736 | 0.095 | 0.701 | 0.005 | 0.837 |
| SAA4 | <0.001 | 0.941 | 0.070 | 0.918 | 0.17 | 0.589 | 0.006 | 0.912 |

The embodiments described above are only to exemplify the present invention but not to limit the scope of the present invention. Any equivalent modification or variation according to the spirit or characteristics of the present invention is to be also included within the scope of the present invention.

What is claimed is:

1. A bladder cancer biomarker, which contains at least one selected from of a group consisting of apolipoprotein A1 (APOA1), apolipoprotein A2 (APOA2), peroxiredoxin 2 (PRDX2), heparin cofactor 2 precursor (HCII), and serum amyloid A-4 protein (SAA4), wherein said bladder cancer biomarker exists in urine of a testee.

2. The bladder cancer biomarker according to claim 1, which is detected with an isobaric tagging for relative and absolute quantification (iTRAQ) technology.

3. A bladder cancer biomarker, which contains at least one selected from the following 69 proteins: Protein S100-P (S100P), Cerulopasmin precursor (SP), Serum amyloid A-4 protein precursor (SAA4), Isoform 1 of Complement factor B precursor (Fragment)(CFB), Afamin precursor (AFM), Apolipoprotein A-I precursor (APOA1), Apolipoprotein A-II precursor (APOA2), Isoform 1 of Fibrinogen alpha chain precursor (FGA), Isoform Gamma-B of Fibrinogen alpha chain precursor (FGG), Apolipoprotein B-100 precursor (APOB), Alpha-1-acid glycolprotein 1 precursor (ORM1), Transthyretin precursor (TTR), ALB protein (ALB), Serotransferrin precursor (TF), Hemopexin precursor (HPX), Antithrombin III variant (SERPINC 1), Angiotensinogen precursor (AGT), 187 kDa protein (C3), FLJ00385 protein (Fragment)(IGHM), Glutathione S-transferase P (GSTP1), Fibrinogen beta chain precursor (FGB), Beta-2-glycoprotein 1 precursor (APOH), Complement C2 precursor (Fragment)(C2), Apolipoprotein A-IV precursor (APOA4), ENO1P protein (EDARAD), Hemoglobin subunit alpha (HBA1; HBA2), Peptidyl-prolyl cis-transisomerase A (PPlA; LOC 654188; LOC 653214), Hemoglobin subunit delta (HBD; HBB), Alpha-2-macroglobulin precursor (A2M), Alpha-1-antitrypsin precursor (SERPINA1), Vitamin D-binding protein precursor (GC), Immunglobulin heavy chain variable region, Myosin-reactive immunoglobulin heavy chain variable region (Fragment), Heparin cofactor 2 precursor (SERPIND1 (HCII)), Peroxiredoxin-2 (PRDX2), heterogeneous nuclear ribonucleoprotein D-like (HNRPDL), Keratin-8-like protein 1, Isoform 2 of Apolipoprotein-L1 precursor (APOL1), Ig heavy chain V-I region EU, Protein S100-A6 (S100A6), Fetuin-B precursor (FETUB), Factor VII active site mutant immunoconjugate (F7), RcTPI1 (Fragment) (LOC729708), Isoform a 1 of Acyl-CoA-binding protein (DBI), IGHA1 protein (IGHA1), Ig heavy chain VIII region GAL, Macrophage migration inhibitory factor (MIF), 14-3-3 protein theta (YWHAQ), Ig mu heavy chain disease protein, HP protein (HP), Serum paraoxonase/arylesterase 1 (PON1), Complement component C9 precursor (C9), Fructose-bisphosphate aldolase A (ALDOA), Kallikrein B, plasma (Fletcher factor) 1 (KLKB1), Inter-alpha-trypsin inhibitor heavy chain H2 precursor (ITIH2), Protein S100-A4 (S100A4), Malate dehydrogenase, mitochondrial precursor (MDH2), Protein S100-A11 (S100A11), Extracellular matrix protein 1 precursor (ECM1), Complement factor H-related protein 3 precursor (CFHR3), Hemoglobin subunit beta (HBB), 101 kDa protein (NDST1), Nucleoside diphosphate kinase A (NME1), Apolipoprotein C-III precursor (APOC3), Histone H4 (HIST1H4), Isoform 1 of Haptoglobin-related protein precursor (HPR), TALDO1 protein (TALDO1), IGHG4 protein (IGHG4), and Myosin-reactive immunoglobulin heavy chain variable region (Fragment), wherein said bladder cancer biomarker exists in urine of a testee.

4. The bladder cancer biomarker according to claim 3, which is detected with an isobaric tagging for relative and absolute quantification (iTRAQ) technology.

5. A method for detecting bladder cancer comprising steps:
providing a urine specimen of a testee;
providing at least one biomarker; and
detecting an expression intensity of said biomarker in said urine specimen, wherein said biomarker is selected from a group consisting of apolipoprotein A1 (APOA1), apolipoprotein A2 (APOA2), peroxiredoxin 2 (PRDX2), heparin cofactor 2 precursor (HCII), and serum amyloid A-4 protein (SAA4).

6. The method for detecting bladder cancer according to claim 5, wherein said expression intensity is obtained via comparing a concentration of said biomarker in said urine specimen of said testee with concentrations of said biomarker in urine specimens of healthy persons.

7. The method for detecting bladder cancer according to claim 5, wherein said expression intensity is obtained via comparing a concentration of said biomarker in said urine specimen of said testee with a concentration of said biomarker in a former urine specimen of said testee.

8. The method for detecting bladder cancer according to claim 5, wherein said expression intensity is an indicator of existence of bladder cancer or an indicator of aggressiveness and/or malignancy of bladder cancer.

9. The method for detecting bladder cancer according to claim 5, wherein content of said biomarker is detected with a western blot method, a mass spectrometric method, a fluorescent method, a luminescent method, an immunological method, or a chromatographic method.

10. The method for detecting bladder cancer according to claim 5, which cooperates with a occult blood test, an NMP22 molecular test, a cystoscopic examination or a cytological examination to improve diagnosis of bladder cancer and evaluation of aggressiveness and malignancy of bladder cancer.

11. A method for detecting bladder cancer comprising steps:
providing a urine specimen of a testee;
providing at least one biomarker; and
detecting an expression intensity of said biomarker in said urine specimen, wherein said biomarker is selected from the following 69 proteins: Protein S100-P (S100P), Cerulopasmin precursor (SP), Serum amyloid A-4 protein precursor (SAA4), Isoform 1 of Complement factor B precursor (Fragment)(CFB), Afamin precursor (AFM), Apolipoprotein A-I precursor (APOA1), Apolipoprotein A-H precursor (APOA2), Isoform 1 of Fibrinogen alpha chain precursor (FGA), Isoform Gamma-B of Fibrinogen alpha chain precursor (FGG), Apolipoprotein B-100 precursor (APOB), Alpha-1-acid glycolprotein 1 precursor (ORM1), Transthyretin precursor (TTR), ALB protein (ALB), Serotransferrin precursor (TF), Hemopexin precursor (HPX), Antithrombin III variant (SERPINC 1), Angiotensinogen precursor (AGT), 187 kDa protein (C3), FLJ00385 protein (Fragment)(IGHM), Glutathione S-transferase P (GSTP1), Fibrinogen beta chain precursor (FGB), Beta-2-glycoprotein 1 precursor (APOH), Complement C2 precursor (Fragment)(C2), Apolipoprotein A-IV precursor (APOA4), ENO1P protein (EDARAD), Hemoglobin subunit alpha (HBA1; HBA2), Peptidyl-prolyl cis-transisomerase A (PPlA; LOC 654188; LOC 653214), Hemoglobin subunit delta (HBD; HBB), Alpha-2-macroglobulin precursor (A2M), Alpha-1-antitrypsin precursor (SERPINA1), Vitamin D-binding protein precursor (GC), Immunglobulin heavy chain variable region, Myosin-reactive immunoglobulin heavy chain variable region (Fragment), Heparin cofactor 2 precursor (SERPIND1 (HCII)), Peroxiredoxin-2 (PRDX2), heterogeneous nuclear ribonucleoprotein D-like (HNRPDL), Keratin-8-like protein 1, Isoform 2 of Apolipoprotein-L1 precursor (APOL1), Ig heavy chain V-I region EU, Protein S100-A6 (S100A6), Fetuin-B precursor (FETUB), Factor VII active site mutant immunoconjugate (F7), RcTPI1 (Fragment) (LOC729708), Isoform a 1 of Acyl-CoA-binding protein (DBI), IGHA1 protein (IGHA1), Ig heavy chain VIII region GAL, Macrophage migration inhibitory factor (MIF), 14-3-3 protein theta (YWHAQ), Ig mu heavy chain disease protein, HP protein (HP), Serum paraoxonase/arylesterase 1 (PON1), Complement component C9 precursor (C9), Fructose-bisphosphate aldolase A (ALDOA), Kallikrein B, plasma (Fletcher factor) 1 (KLKB1), Inter-alpha-trypsin inhibitor heavy chain H2 precursor (ITIH2), Protein S100-A4 (S100A4), Malate dehydrogenase, mitochondrial precursor (MDH2), Protein S100-A11 (S100A11), Extracellular matrix protein 1 precursor (ECM1), Complement factor H-related protein 3 precursor (CFHR3), Hemoglobin subunit beta (HBB), 101 kDa protein (NDST1), Nucleoside diphosphate kinase A (NME1), Apolipoprotein C-III precursor (APOC3), Histone H4 (HIST1H4), Isoform 1 of Haptoglobin-related protein precursor (HPR), TALDO1 protein (TALDO1), IGHG4 protein (IGHG4), and Myosin-reactive immunoglobulin heavy chain variable region (Fragment), wherein said bladder cancer biomarker exists in urine of a testee.

12. The method for detecting bladder cancer according to claim 11, wherein said expression intensity is obtained via comparing a concentration of said biomarker in said urine specimen of said testee with concentrations of said biomarker in urine specimens of healthy persons.

13. The method for detecting bladder cancer according to claim 11, wherein said expression intensity is obtained via comparing a concentration of said biomarker in said urine specimen of said testee with a concentration of said biomarker in a former urine specimen of said testee.

14. The method for detecting bladder cancer according to claim 11, wherein said expression intensity is an indicator of existence of bladder cancer or an indicator of aggressiveness and/or malignancy of bladder cancer.

15. The method for detecting bladder cancer according to claim 11, wherein content of said biomarker is detected with a western blot method, a mass spectrometric method, a fluorescent method, a luminescent method, an immunological method, or a chromatographic method.

16. The method for detecting bladder cancer according to claim 11, which cooperates with a occult blood test, an MNP22 molecular test, a cystoscopic examination or a cytological examination to improve diagnosis of bladder cancer and evaluation of aggressiveness and malignancy of bladder cancer.
